(19) United States
(12) Patent Application Publication
(10) Pub. No.: US 2008/0072914 A1
(43) Pub. Date: Mar. 27, 2008

(54) Bronchial Isolation Devices for Placement in Short Lumens

(76) Inventors: Michael J. Hendricksen, Redwood City, CA (US); Antony J. Fields, San Francisco, CA (US); Michael Barrett, Campbell, CA (US); Ronald R. Hundertmark, San Mateo, CA (US); Alan R. Rapacki, Redwood City, CA (US); Steve Wallace, Tracy, CA (US); Michael Regan, Pleasanton, CA (US); John G. McCutcheon, Menlo Park, CA (US)

Correspondence Address: FISH & RICHARDSON, PC, P.O. BOX 1022, MINNEAPOLIS, MN, 55440-1022, US

(21) Appl. No.: 11/844,700

(22) Filed: Aug. 24, 2007

Related U.S. Application Data
(60) Provisional application No. 60/840,128, filed on Aug. 25, 2006.

(51) Int. Cl. A62B 9/02; A61F 2/04

(52) U.S. Cl. 128/207.16; 623/23.68

(57) ABSTRACT

Methods and devices are adapted for regulating fluid flow to and from a region of a patient's lung, such as to achieve a desired fluid flow dynamic to a lung region during respiration and/or to induce collapse in one or more lung regions. Pursuant to an exemplary procedure, an identified region of the lung is targeted for treatment. The targeted lung region is then bronchially isolated to regulate airflow into and/or out of the targeted lung region through one or more bronchial passageways that feed air to the targeted lung region.

### REFERENCE TO PRIORITY DOCUMENT

This application claims priority of co-pending U.S. Provisional Patent Application Ser. No. 60/840,128 filed Aug. 25, 2006. Priority of the aforementioned filing date is hereby claimed and the disclosure of the Provisional patent application is hereby incorporated by reference in its entirety.

### BACKGROUND

Pulmonary diseases, such as chronic obstructive pulmonary disease, (COPD), reduce the ability of one or both lungs to fully expel air during the exhalation phase of the breathing cycle. Such diseases are accompanied by chronic or recurrent obstruction to air flow within the lung. Because of the increase in environmental pollutants, cigarette smoking, and other noxious exposures, the incidence of COPD has increased dramatically in the last few decades and now ranks as a major cause of activity-restricting or bed-confining disability in the United States. COPD can include such disorders as chronic bronchitis, bronchiectasis, asthma, and emphysema.

It is known that emphysema and other pulmonary diseases reduce the ability of one or both lungs to fully expel air during the exhalation phase of the breathing cycle. One of the effects of such diseases is that the diseased lung tissue is less elastic than healthy lung tissue, which is one factor that prevents full exhalation of air. During breathing, the diseased portion of the lung does not fully recoil due to the diseased (e.g., emphysematic) lung tissue being less elastic than healthy tissue. Consequently, the diseased lung tissue exerts a relatively low driving force, which results in the diseased lung expelling less air volume than a healthy lung. The reduced air volume exerts less force on the airway, which allows the airway to close before all air has been expelled, another factor that prevents full exhalation.

The problem is further compounded by the diseased, less elastic tissue that surrounds the very narrow airways that lead to the alveoli, which are the air sacs where oxygen-carbon dioxide exchange occurs. The diseased tissue has less tone than healthy tissue and is typically unable to maintain the narrow airways open until the end of the exhalation cycle. This traps air in the lungs and exacerbates the already-inefficient breathing cycle. The trapped air causes the tissue to become hyper-expanded and no longer able to effect efficient oxygen-carbon dioxide exchange.

In addition, hyper-expanded, diseased lung tissue occupies more of the pleural space than healthy lung tissue. In most cases, a portion of the lung is diseased while the remaining part is relatively healthy and, therefore, still able to efficiently carry out oxygen exchange. By taking up more of the pleural space, the hyper-expanded lung tissue reduces the amount of space available to accommodate the healthy, functioning lung tissue. As a result, the hyper-expanded lung tissue causes inefficient breathing due to its own reduced functionality and because it adversely affects the functionality of adjacent healthy tissue.

Some recent treatments include the use of devices that isolate a diseased region of the lung in order to reduce the volume of the diseased region, such as by collapsing the diseased lung region. According to such treatments, one or more flow control devices are implanted in airways feeding a diseased region of the lung to regulate fluid (gas or liquid) flow to the diseased lung region in order to fluidly isolate the region of the lung. These implanted flow control devices can be, for example, one-way valves that allow flow in the exhalation direction only, occluders or plugs that prevent flow in either direction, or two-way valves that control flow in both directions. However, such devices are still in the development stages.

### SUMMARY

In view of the foregoing, there is a need for improvement in the design and functionality of such flow control devices.

In one aspect, there is disclosed a flow control device for a bronchial passageway, comprising: a valve member that regulates fluid flow through the flow control device; a frame coupled to the valve member, the frame including: (a) a distal retainer region being formed of a plurality of interconnected struts configured to engage an interior wall of the bronchial passageway to retain the flow control device in a fixed location therein, the retainer region being movable from a contracted state suitable for introduction into the bronchial passageway to an expanded state suitable for engaging the interior wall of the bronchial passageway; and (b) at least one stabilization structure having a tip that extends distally past a distal edge of the distal retainer region, the stabilization structure sized and shaped to achieve stabilization of the position of the device in the bronchial passageway, wherein the stabilization structures rest against and do penetrate the bronchial wall; and a membrane covering at least a portion of the retainer region, wherein at least a portion of the flow control device forms a seal with the interior wall of the bronchial passageway when the flow control device is implanted in the bronchial passageway.

In another aspect, there is disclosed a flow control device for a bronchial passageway, comprising: a valve member that regulates fluid flow through the flow control device; a frame coupled to the valve member, the frame including: (a) a distal retainer region formed of a plurality of interconnected struts configured to engage an interior wall of the bronchial passageway to retain the flow control device in a fixed location therein, the retainer region being movable from a contracted state suitable for introduction into the bronchial passageway to an expanded state suitable for engaging the interior wall of the bronchial passageway, wherein the distal retainer region includes an elongated section that is sufficiently long to extend into a distal bronchial passageway that branches from a bronchial passageway in which the device is implanted; and a membrane covering at least a portion of the distal retainer region not including the elongate section, wherein at least a portion of the flow control device forms a seal with the interior wall of the bronchial passageway when the flow control device is implanted in the bronchial passageway.

In another aspect, there is disclosed a method of implanting a fluid flow control device in a bronchial passageway, comprising: providing a flow control device having a valve, a retainer, and a seal; and implanting the flow control device in a bronchial passageway such that a proximal portion of the retainer anchors against a wall of a first bronchial passageway and a distal portion of the retainer is positioned in a second bronchial passageway that branches from the first bronchial passageway, wherein the seal covers only the proximal portion of the bronchial passageway.

In another aspect, there is disclosed a flow control device for a bronchial passageway, comprising: a frame having a first end and a second end, the frame defining an internal lumen having a first opening at the first end and a second opening at the second end that both communicate with the lumen; a seal covering at least a portion of the frame; and a valve positioned on the frame between the first end and the second end, wherein the valve regulates fluid flow into the lumen at a location between the first end and the second end

In another aspect, there is disclosed a flow control device for a bronchial passageway, comprising: a first frame adapted to anchor against a wall of a first bronchial passageway, the first frame defining a lumen through which fluid can flow; a seal member coupled to the first frame and adapted to seal against the first bronchial passageway; valve member that regulates fluid flow through the lumen, the valve member coupled to the first frame; a second frame adapted to anchor against a wall of a second bronchial passageway; and a tether connecting the first frame to the second frame

Other features and advantages should be apparent from the following description of various embodiments, which illustrate, by way of example, the principles of the invention.

### BRIEF DESCRIPTION OF THE DRAWINGS

FIG. 1A shows an anterior view of a pair of human lungs and a bronchial tree with a bronchial isolation device implanted in a bronchial passageway to bronchially isolate a region of the lung.

FIG. 1B shows a representative one-way valve bronchial isolation device.

FIG. 1C shows examples of stable and unstable bronchial isolation device placements.

FIG. 2A illustrates an anterior view of a pair of human lungs and a bronchial tree.

FIG. 2B illustrates a lateral view of the right lung.

FIG. 2C illustrates a lateral view of the left lung.

FIG. 2D illustrates an anterior view of the trachea and a portion of the bronchial tree

FIG. 3 shows a first embodiment of a bronchial isolation device.

FIG. 4 shows another embodiment of a bronchial isolation device mounted in a bronchial passageway.

FIG. 5 shows a perspective view of the device of FIG. 4.

FIG. 6A shows another embodiment of a bronchial isolation device mounted in a bronchial passageway.

FIG. 6B shows a perspective view of the device of FIG. 6A.

FIG. 7A shows another embodiment of a bronchial isolation device mounted in a bronchial passageway.

FIG. 7B shows a perspective view of the device of FIG. 7A

FIG. 8 shows another embodiment of a bronchial isolation device mounted in a bronchial passageway.

FIG. 9 shows another embodiment of a bronchial isolation device mounted in a bronchial passageway.

FIG. 10 shows a perspective view of the device of FIG. 9.

FIG. 11 shows another embodiment of a bronchial isolation device mounted in a bronchial passageway.

FIG. 12 shows another embodiment of a bronchial isolation device mounted in a bronchial passageway.

FIG. 13 shows another embodiment of a bronchial isolation device mounted in a bronchial passageway.

FIG. 14 shows a perspective view of the device of FIG. 13.

FIG. 15 shows another embodiment of a bronchial isolation device mounted in a bronchial passageway.

FIG. 16 shows a perspective view of the device of FIG. 15.

FIG. 17A shows another embodiment of a bronchial isolation device mounted in a bronchial passageway.

FIG. 17B shows another embodiment of a bronchial isolation device mounted in a bronchial passageway.

FIG. 18 shows another embodiment of a bronchial isolation device mounted in a bronchial passageway.

FIG. 19 shows a perspective view of the device of FIG. 18.

FIGS. 20 and 21 show exemplary embodiments of an expansion ring of the device of FIGS. 18 and 19.

### DETAILED DESCRIPTION

Disclosed are methods and devices for regulating fluid flow to and from a region of a patient's lung, such as to achieve a desired fluid flow dynamic to a lung region during respiration and/or to induce collapse in one or more lung regions. Pursuant to an exemplary procedure, an identified region of the lung (referred to herein as the “targeted lung region”) is targeted for treatment. The targeted lung region is then bronchially isolated to regulate airflow into and/or out of the targeted lung region through one or more bronchial passageways that feed air to the targeted lung region.

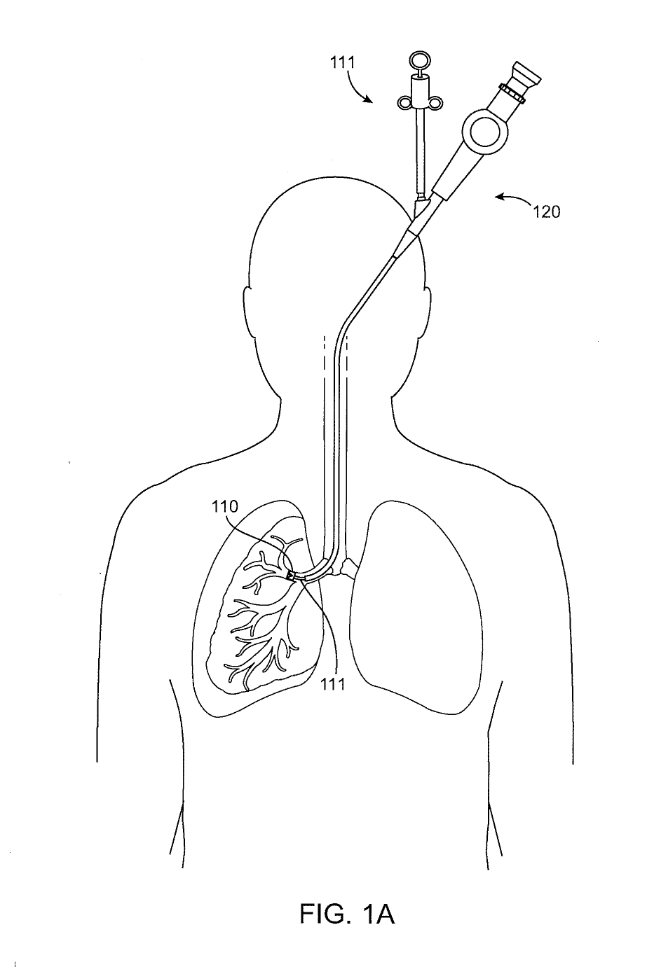

As shown in FIG. 1A, the bronchial isolation of the targeted lung region is accomplished by implanting a flow control device 110 (sometimes referred to as a bronchial isolation device) into a bronchial passageway that feeds air to a targeted lung region. The flow control device 110 regulates fluid flow through the bronchial passageway (sometimes referred to as a bronchial lumen) in which the flow control device 110 is implanted. The flow control device 110 can regulate airflow through the bronchial passageway 115 using a valve that permits fluid flow in a first direction (e.g., the exhalation direction) while limiting or preventing fluid flow in a second direction (e.g., the inhalation direction).

With reference still to FIG. 1A, the bronchial isolation device 110 is delivered into the lung to by mounting the device 110 at the distal end of a delivery catheter 111 and then inserting the delivery catheter into the bronchial passageway. Once the distal end is properly is positioned in the bronchial passageway, the bronchial isolation device 110 is ejected from the delivery catheter 111 and deployed within the passageway. In the example shown in FIG. 1A, the distal end of the delivery catheter 111 is inserted into the patient's mouth or nose, through the trachea, and down to a target location in the bronchial passageway using a bronchoscope 120. Alternately, the delivery catheter 111 can be guided to the target location in the patient's lungs using a guidewire.

Bronchial isolation devices are often designed to be self-expanding so that once they are deployed in a bronchial passageway (i.e., the airway), the bronchial isolation devices self-expand to fill the bronchial passageway and grip the bronchial wall. In order for these devices that are retained in the passageway by self-expanding to be stable in the airway after deployment, the length of engagement with the bronchial wall is desirably greater than the diameter of the passageway. If the diameter is greater than the passageway length, the device can move or rotate in an uncontrollable fashion away from the implant location.

FIG. 1B shows a representative one-way valve bronchial isolation device. The device includes a self-expanding retainer 10 (such as a Nitinol retainer) which is covered in a seal member such as a silicone membrane 11. The retainer 10 is comprised of a plurality of interconnected struts that collectively form the outer periphery of the bronchial isolation device. The retainer 10 is laser cut from tubing, such as Nitinol tubing, and expanded and heat treated to the shape shown in FIG. 1B. The retainer can also be made of woven Nitinol wire or by and other manufacturing technique that would allow the retainer 10 to be compressed for insertion and that will resiliently expand once released to grip the bronchial wall.

The retainer 10 includes a retainer portion 13 and a valve protector 14. The retainer portion 10 has a diameter that is larger than the diameter of the valve protector 14. When the bronchial isolation device is deployed within a bronchial passageway, the diameter of the retainer portion is sufficiently large to cause the retainer portion to press against and anchor to the walls of the bronchial passageway to secure the bronchial isolation device in a fixed location relative to the bronchial passageway. The retainer can transition between a contracted state and an expanded state. In the contracted state, the retainer has a diameter that is smaller than the diameter of the retainer in the expanded state.

A silicone one-way duckbill valve 12 is bonded to the membrane 11 inside the valve protector 14. The valve protector 14 is adapted to prevent the valve 12 from being distorted by the bronchial wall during cough and other events that constrict the bronchial passageway. The distal larger diameter portion of the self-expanding retainer 10 is the distal retainer 14, which expands to come in full contact with and to grip the bronchial wall after implantation. The deformable membrane 11 is sealed against the bronchial wall due to the outward expansion of the self-expanding retainer 10. The seal prevents inhaled air from flowing past the device in the distal direction (indicated by the arrow labeled 15) during inhalation. The one-way valve 12 allows air to vent through the valve in a proximal direction (indicated by the arrow labeled 16) during exhalation. This device could also be modified to be an occluder, or a two-way valve instead of the one-way valve that is shown in the figure. In addition, the retainer can be manufactured of other self-expanding materials other than Nitinol, and the valve and membrane can be manufactured from deformable materials other than silicone such as urethane.

In an embodiment, the length of the distal retainer 15 is desirably greater than the diameter of the bronchial passageway, or the device may be unstable in the airway. Stable and unstable bronchial isolation device placements are shown in FIG. 1C. An unstable bronchial isolation device 21 is shown implanted in a short passageway where the diameter of the passageway D1 is greater than the length of the passageway L1. A stable bronchial isolation device 22 is shown implanted in a longer passageway where the diameter of the passageway D2 is less than the length of the passageway L2.

Given that it is desirable to implant bronchial isolation devices into any bronchial passageway in the lungs, there are instances where it is desirable to implant a bronchial isolation device in a passageway where the bronchial passageway length is less than the diameter. In these cases, a standard bronchial isolation device similar to that shown in FIG. 1B may be unsuitable. What has been needed are implantable bronchial isolation devices that are designed to be stable in bronchial passageways where the diameter is greater than the length, while still being resistant to migration distally or proximally.

Disclosed below are various embodiments of bronchial isolation devices for placement in the lungs in difficult locations, such as in bronchial passageways where the length of the passageway is the same as or shorter than the diameter of the passageway. Such devices are stable and resistive to migration or rotation after implantation in bronchial passageways where the length of the passageway is the same as or shorter than the diameter of the passageway. The embodiments described below and shown in the figures are one-way valve bronchial isolation devices. However, they could also be constructed as either occluder or two-way valve bronchial isolation devices.

Exemplary Lung Anatomy

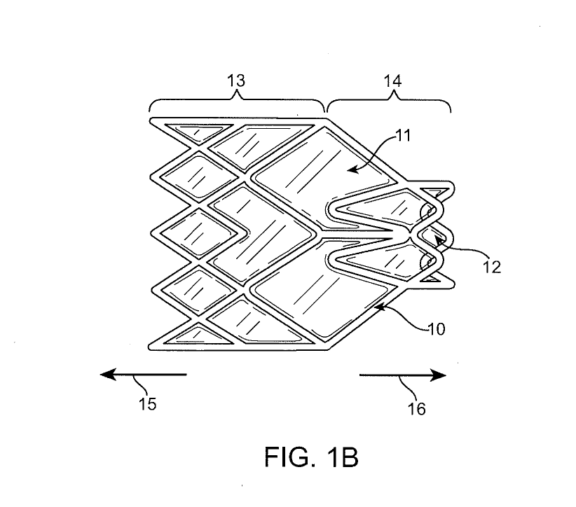

Prior to describing the exemplary embodiments of bronchial isolation devices for placement in the lungs in difficult locations, a general discussion of lung anatomy is provided. FIG. 2A shows an anterior view of a pair of human lungs 210, 215 and a bronchial tree 220 that provides a fluid pathway into and out of the lungs 210, 215 from a trachea 225, as will be known to those skilled in the art. As used herein, the term “fluid” can refer to a gas, a liquid, or a combination of gas(es) and liquid(s). For clarity of illustration, FIG. 2A shows only a portion of the bronchial tree 220, which is described in more detail below with reference to FIG. 2D.

Throughout this description, certain terms are used that refer to relative directions or locations along a path defined from an entryway into the patient's body (e.g., the mouth or nose) to the patient's lungs. The path of airflow into the lungs generally begins at the patient's mouth or nose, travels through the trachea into one or more bronchial passageways, and terminates at some point in the patient's lungs. For example, FIG. 2A shows a path 202 that travels through the trachea 225 and through a bronchial passageway into a location in the right lung 210. The term “proximal direction” refers to the direction along such a path 202 that points toward the patient's mouth or nose and away from the patient's lungs. In other words, the proximal direction is generally the same as the expiration direction when the patient breathes. The arrow 204 in FIG. 2A points in the proximal or expiratory direction. The term “distal direction” refers to the direction along such a path 202 that points toward the patient's lung and away from the mouth or nose. The distal direction is generally the same as the inhalation or inspiratory direction when the patient breathes. The arrow 206 in FIG. 2A points in the distal or inhalation direction.

The lungs include a right lung 210 and a left lung 215. The right lung 210 includes lung regions comprised of three lobes, including a right upper lobe 230, a right middle lobe 235, and a right lower lobe 240. The lobes 230, 235, 240 are separated by two interlobar fissures, including a right oblique fissure 226 and a right transverse fissure 228. The right oblique fissure 226 separates the right lower lobe 240 from the right upper lobe 230 and from the right middle lobe 235. The right transverse fissure 228 separates the right upper lobe 230 from the right middle lobe 235.

As shown in FIG. 2A, the left lung 215 includes lung regions comprised of two lobes, including the left upper lobe 250 and the left lower lobe 255. An interlobar fissure comprised of a left oblique fissure 245 of the left lung 215 separates the left upper lobe 250 from the left lower lobe 255. The lobes 230, 235, 240, 250, 255 are directly supplied air via respective lobar bronchi, as described in detail below.

FIG. 2B is a lateral view of the right lung 210. The right lung 210 is subdivided into lung regions comprised of a plurality of bronchopulmonary segments. Each bronchopulmonary segment is directly supplied air by a corresponding segmental tertiary bronchus, as described below. The bronchopulmonary segments of the right lung 210 include a right apical segment 310, a right posterior segment 320, and a right anterior segment 330, all of which are disposed in the right upper lobe 230. The right lung bronchopulmonary segments further include a right lateral segment 340 and a right medial segment 350, which are disposed in the right middle lobe 235. The right lower lobe 240 includes bronchopulmonary segments comprised of a right superior segment 360, a right medial basal segment (which cannot be seen from the lateral view and is not shown in the figures), a right anterior basal segment 380, a right lateral basal segment 390, and a right posterior basal segment 395.

FIG. 2C shows a lateral view of the left lung 215, which is subdivided into lung regions comprised of a plurality of bronchopulmonary segments. The bronchopulmonary segments include a left apical segment 410, a left posterior segment 420, a left anterior segment 430, a left superior lingular segment 440, and a left inferior lingular segment 450, which are disposed in the left lung upper lobe 250. The lower lobe 255 of the left lung 215 includes bronchopulmonary segments comprised of a left superior segment 460, a left medial basal segment (which cannot be seen from the lateral view and is not shown in the figures), a left anterior basal segment 480, a left lateral basal segment 490, and a left posterior basal segment 495.

FIG. 2D shows an anterior view of the trachea 325 and a portion of the bronchial tree 220, which includes a network of bronchial passageways, as described below. The trachea 225 divides at a lower end into two bronchial passageways comprised of primary bronchi, including a right primary bronchus 510 that provides direct air flow to the right lung 210, and a left primary bronchus 515 that provides direct air flow to the left lung 215. Each primary bronchus 510, 515 divides into a next generation of bronchial passageways comprised of a plurality of lobar bronchi. The right primary bronchus 510 divides into a right upper lobar bronchus 517, a right middle lobar bronchus 520, and a right lower lobar bronchus 422. The left primary bronchus 415 divides into a left upper lobar bronchus 525 and a left lower lobar bronchus 530. Each lobar bronchus 517, 520, 522, 525, 530 directly feeds fluid to a respective lung lobe, as indicated by the respective names of the lobar bronchi. The lobar bronchi each divide into yet another generation of bronchial passageways comprised of segmental bronchi, which provide air flow to the bronchopulmonary segments discussed above.

As is known to those skilled in the art, a bronchial passageway defines an internal lumen through which fluid can flow to and from a lung or lung region. The diameter of the internal lumen for a specific bronchial passageway can vary based on the bronchial passageway's location in the bronchial tree (such as whether the bronchial passageway is a lobar bronchus or a segmental bronchus) and can also vary from patient to patient. However, the internal diameter of a bronchial passageway is generally in the range of 3 millimeters (mm) to 10 mm, although the internal diameter of a bronchial passageway can be outside of this range. For example, a bronchial passageway can have an internal diameter of well below 1 mm at locations deep within the lung. The internal diameter can also vary from inhalation to exhalation as the diameter increases during inhalation as the lungs expand, and decreases during exhalation as the lungs contract.

Throughout this disclosure, reference is sometimes made to a “direct pathway” to a targeted lung region and to a “collateral pathway” (or simply a “collateral”) to a targeted lung region. The term “direct pathway” refers to a bronchial passageway that branches directly or indirectly from the trachea and either

(1) terminates in the targeted lung region to thereby directly provide air to the targeted lung region; or (2) branches into at least one other bronchial passageway that terminates in the targeted lung region to thereby directly provide air to the targeted lung region. The term “collateral pathway” (or simply a “collateral”) refers to any pathway that provides air to the targeted lung region and that is not a direct pathway.

The term “direct’ is used to refer to air flow that flows into or out of a targeted lung region via a direct pathway. Likewise, the term “collateral” is used to refer to fluid flow (such as air flow) that flows into or out of a targeted lung region via a collateral pathway. Thus, for example, “direct” flow is fluid flow (such as air flow) that enters or exits the targeted lung region via a direct pathway, and “collateral” flow is fluid flow (such as air flow) that enters or exits the targeted lung region via a collateral pathway. A collateral flow can be, for example, air flow that flows between segments of a lung, which is referred to as intralobar flow, or it can be, for example, air flow that flows between lobes of a lung, which is referred to as interlobar flow.

Exemplary Bronchial Isolation Devices

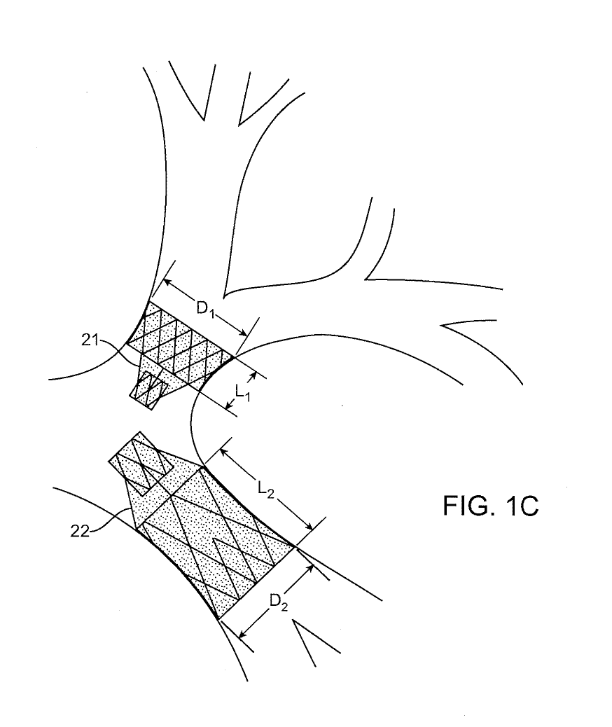

FIG. 3 shows a first embodiment of a bronchial isolation device. The device, which is similar to that shown in FIG. 1B, comprises a self-expanding retainer 30, a deformable seal member such as membrane 31, a one-way valve 32, and valve protector section 34 and a distal retainer section 33. In order to keep the device stable in the airway, one or more stabilization barbs 35 are disposed at or near a distal end of the distal retainer 33. The barbs 35 are prongs that extend outward from a region of the device.

The barbs 35 sink into and anchor with the tissue of the bronchial passageway wall and keep the device from migrating or rotating. The barbs 35 can be located on the proximal end of the distal retainer 33, in-between the proximal and distal ends, or in any other location that would allow them to sink into the bronchial passageway wall tissue and stabilize the device. In an embodiment, the barbs 35 have tips that extend past the distal end of the distal retainer 33.

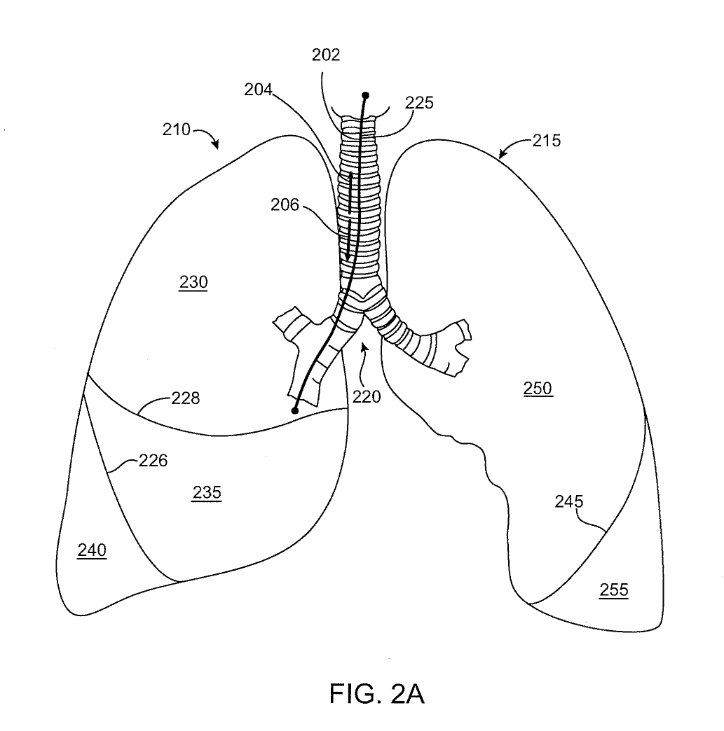

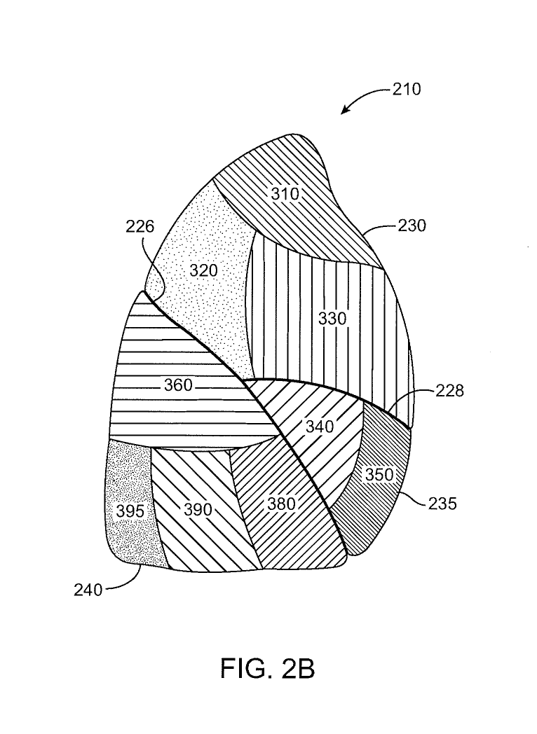

In another embodiment shown in FIGS. 4 and 5, a bronchial isolation device similar to that shown in FIG. 1B is shown. FIG. 4 shows the device mounted in a bronchial passageway and FIG. 5 shows a perspective view of the device. The device comprises a self-expanding retainer 40, a deformable membrane 41, a one-way valve 42, and valve protector section 44 and a distal retainer section 43. The device includes one or more sharp ridges 45 that are disposed around the outside of the circumference of the retainer, such as in an annular fashion. The ridges 45 can extend entirely around the circumference of the device or a plurality of ridges can be interspersed throughout the diameter. The ridges function to keep the device stable in the airway.

When the device is implanted in a bronchial passageway, the ridge 45 sinks into or anchors with the tissue of the bronchial passageway and prevents the device from migrating or rotating inside the bronchial passageway. The retainer can be comprised of a single ridge or two or more ridges, and they can be located anywhere along the length of the distal retainer 43. In addition, the ride 45 can be integrally formed with the retainer or it can be a separate piece that is bonded to the retainer.

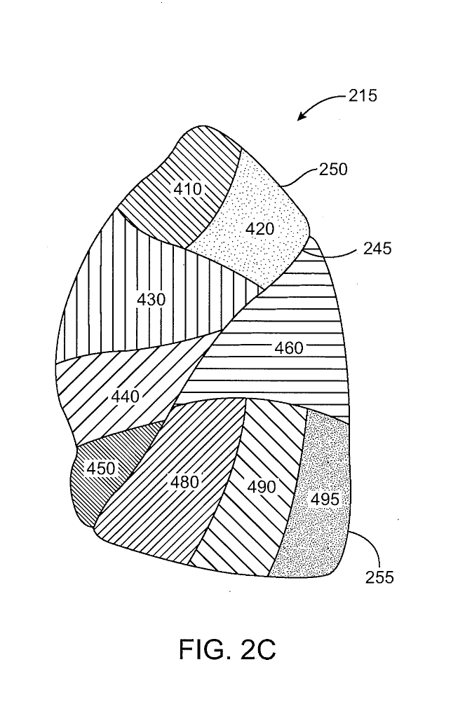

FIGS. 6A and 6B show yet another embodiment of a bronchial isolation device. The device comprises a self-expanding retainer 60, a deformable membrane 61, a one-way valve 62, and valve protector section 64 and a distal retainer section 63. In order to keep the device stable in the airway, the distal retainer 63 includes one or more stabilizing arms 65 that extend distally from the distal edge 66 of the distal retainer 63. The stabling arms are sized and shaped to rest along the surface of the bronchial wall. The stabilizing arms can be any shape or size that is adapted to achieve stabilization of the position of the device in the bronchial passageway. The stabilizing arm or arms are biased towards the bronchial wall and rest along the surface of the wall to stabilize the device in the airway. Unlike the stabilization barbs 35 of the embodiment shown in FIG. 3, the stabilizing arms 65 do not penetrate the tissue of the bronchial wall. In an embodiment, the stabilizing arms are sufficiently long to extend into a distal carina.

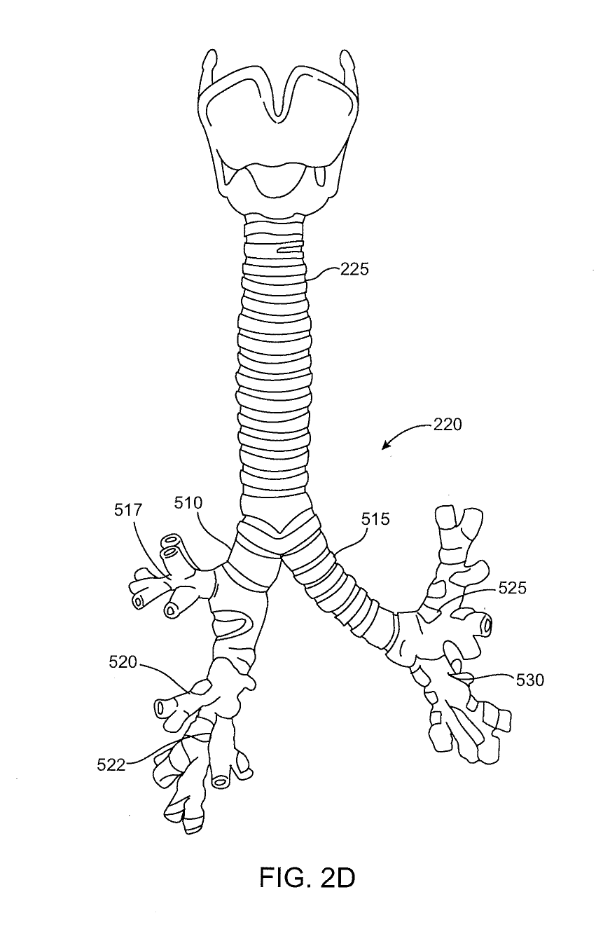

FIGS. 7A and 7B show yet another embodiment of a bronchial isolation device that is similar to the device shown in FIGS. 6A and 6B. The device comprises a self-expanding retainer 70, a deformable membrane 71, a one-way valve 72, and valve protector section 74 and a distal retainer section 73. As in the previous embodiment, the distal retainer 73 includes stabilizing arms 75 that extend distally from the distal edge of the distal retainer 73. In the embodiment of FIGS. 7A and 7B, the stabilizing arms are interspersed around the circumference of the distal edge of the distal retainer 73. The stabilizing arms 75 have atraumatic tips, such as rounded or curved tips or any other configuration that is atraumatic. The atraumatic tips permit the device to be pushed up to the carina such that the stabilizing arms 75 easily move to one side or the other of the carina. The stabilizing arms 75 lodge against various locations in the region of the carina to stabilize the device without requiring rotational orientation of the arms relative to the carina. In an embodiment, the stabilizing arms are sufficiently long to extend into a distal carina.

In any of the embodiments of FIGS. 6A-7B, the stabling arms are sized and shaped to rest along the surface of the bronchial wall. The stabilizing arms can be any shape or size that is adapted to achieve stabilization of the position of the device in the bronchial passageway. The stabilizing arm or arms are biased towards the bronchial wall and rest along the surface of the wall to stabilize the device in the airway. Unlike the stabilization barbs 35 of the embodiment shown in FIG. 3, the stabilizing arms 65 and 75 do not penetrate the tissue of the bronchial wall.

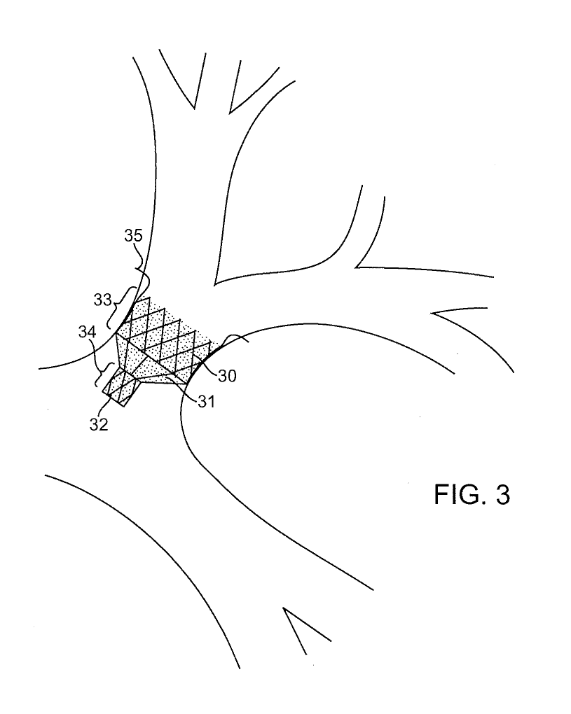

FIG. 8 shows yet another embodiment of a bronchial isolation device. The device is similar to that shown in FIG. 1B and comprises a self-expanding retainer 80, a deformable membrane 81, a one-way valve 82, and valve protector section 84 and a distal retainer section 83. In order to keep the device stable in the airway, the distal retainer portion 83 has a length that is sufficiently long to extend into one of the next most distal bronchial passageways that branch from the passageway in which the device is implanted into. The elongated distal retainer portion increases the effective length of the device and stabilizes the device in the airway. Unlike the embodiment shown in FIG. 1B where the distal retainer portion 13 is completely covered with the deformable membrane 11, the distal retainer 83 is only partially covered by the deformable membrane 81.

In an embodiment, a proximal portion 85 of the distal retainer 83 is covered by the deformable membrane 81, and a distal portion 86 of the distal retainer 83 is not covered by the deformable membrane 81. Exhaled air can flow through the center of the device from the bronchial passageway in which the distal portion of the device is implanted in (as shown by arrow 87), through the one-way valve 82 and out of the lungs towards the trachea (as shown by arrow 89). In addition, exhaled air can flow through the open areas of the distal portion 86 of the distal retainer 83 (as shown by arrow 88) that is not covered by the deformable membrane 81, through the one-way valve 82 and out of the lungs towards the trachea (as shown by arrow 89). The deformable membrane 81 that covers the proximal portion 85 of the distal retainer 83 seals the device against the bronchial passageway wall to prevent air from flowing past the device during inhalation.

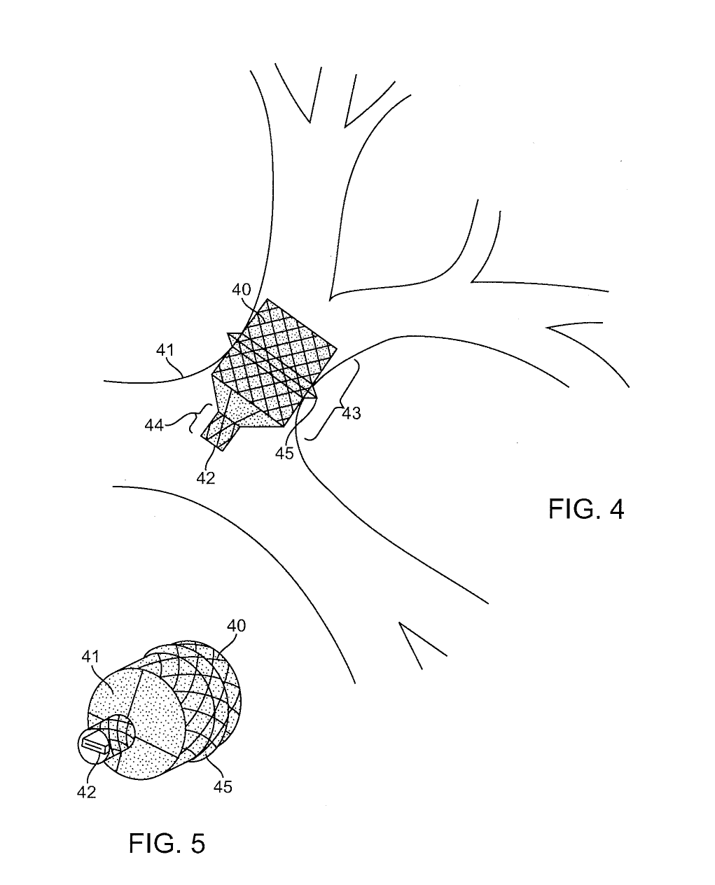

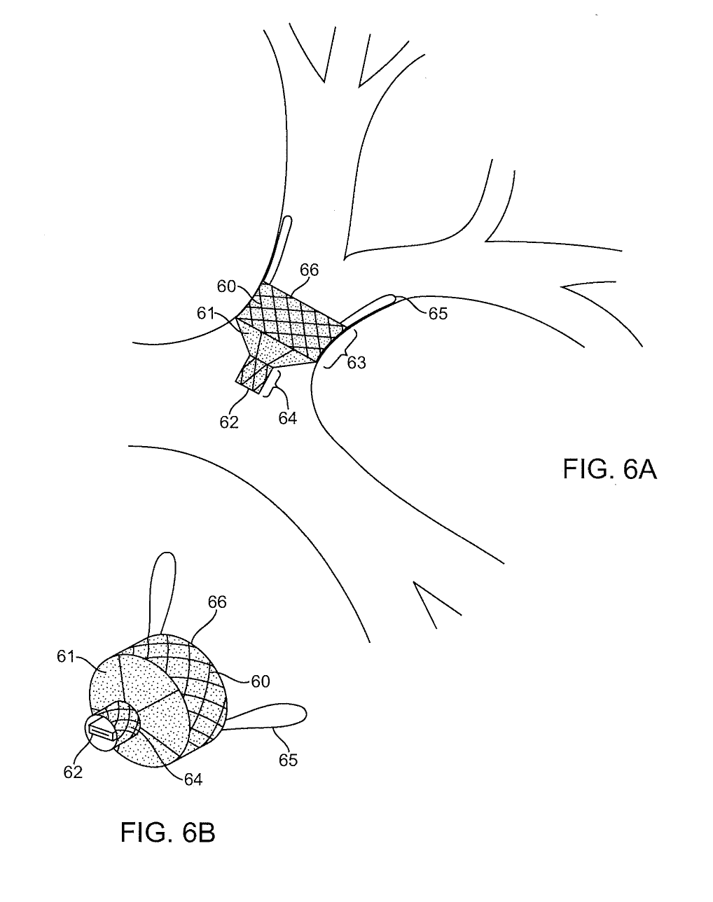

FIGS. 9 and 10 show yet another embodiment of a bronchial isolation device, which is similar to that shown in FIG. 1B. The device comprises a self-expanding retainer 90, a deformable membrane 91, a one-way valve 92, and valve protector section 94 and a distal retainer section 93. In order to keep the device stable in the airway, the distal retainer 93 is flared at the proximal end 95 and at the distal end 96, and has a smaller diameter between the proximal end 95 and at the distal end 96. This provides the device with an hourglass-type shape although it should be appreciated that the shape can vary as long as the device is wider at the proximal and distal ends than in the middle. The shape of the device permits the device to conform to the shape of a short bronchial passageway.

When implanted in a bronchial passageway that is too short for devices such as that shown in FIG. 1B, the distal retainer flares 95 and 96 grips the airway on the proximal and distal end to stabilize the device. Retention prongs 35 such as those shown in FIG. 3 may be added to the proximal flare 95, to the distal flare 96, to both, or in-between the proximal and distal flares 95 and 96 as needed to improve retention and stability.

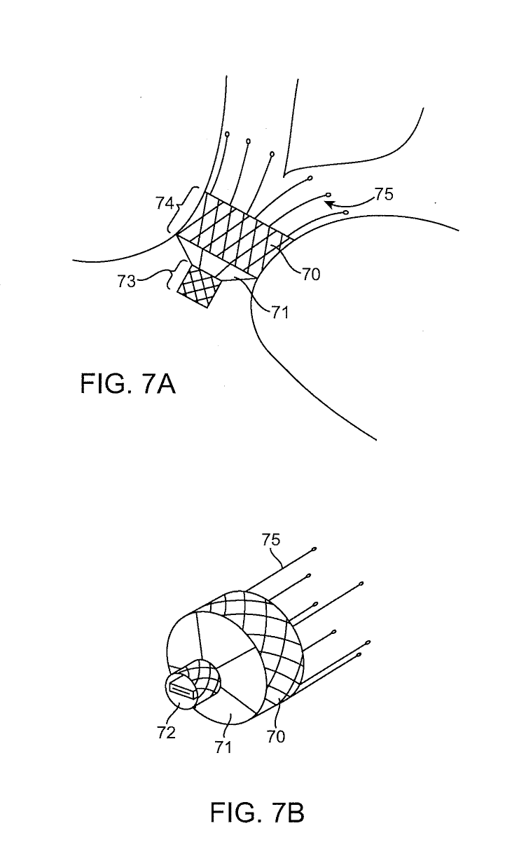

In yet another embodiment shown in FIG. 11, a bronchial isolation device 111, similar to that shown in FIG. 1B, is shown implanted inside a tubular stent 113 that is itself implanted in the bronchial passageway. Without being coupled to the stent 113, the bronchial isolation device 111 would be unstable in the short bronchial passageway shown in FIG. 11. However the tubular stent 113 is implanted in the bronchial passageway to create a stable, smooth and longer passageway. The tubular stent can be made of solid silicone or other elastomer, it can be manufactured of Nitinol or other metal in order to be self-expanding, or of any other material or combination of materials that is deformable so that the device can expand into contact with the bronchial passageway wall. Once the tubular stent 113 is implanted in the bronchial passageway, the stent 113 creates a stable passageway in which the bronchial isolation device can be implanted. A standard bronchial isolation device 111 is then implanted inside the tubular stent 113.

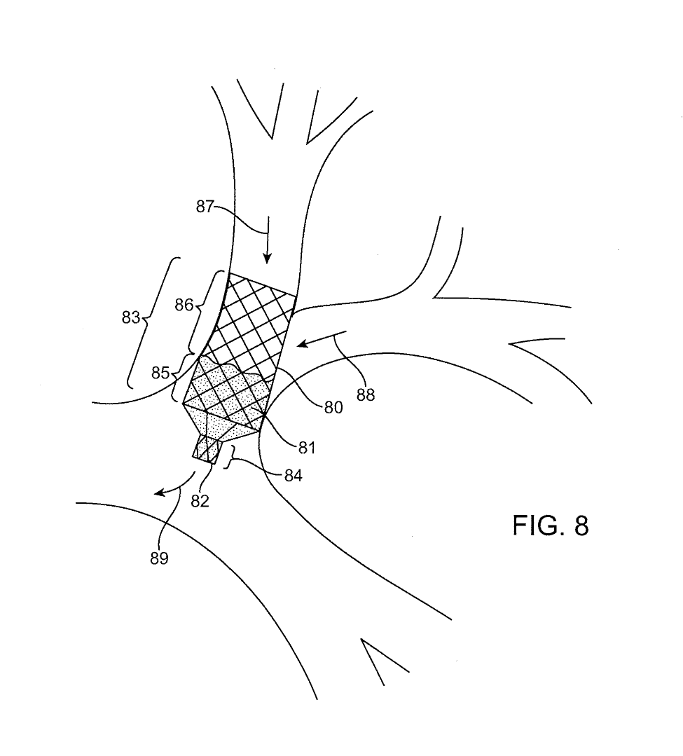

FIG. 12 shows yet another embodiment of a bronchial isolation device. The device is similar to that shown in FIG. 1B and comprises a self-expanding retainer 120, a deformable membrane 121, a one-way valve 122, and valve protector section 124 and a distal retainer section 123. The distal retainer section 123 bifurcates into two or more legs that are each sized and shaped to fit into a branch of the bronchial passageway. Thus, in order to keep the device stable in the airway, the distal retainer portion 123 is bifurcated into two legs 125 and 126, such as the legs of a pair of pants. The two legs 125 and 126 each extend down one of the two bronchial passageways that branch distally from the bronchial passageway that the device is implanted in, thus stabilizing and retaining the device in place.

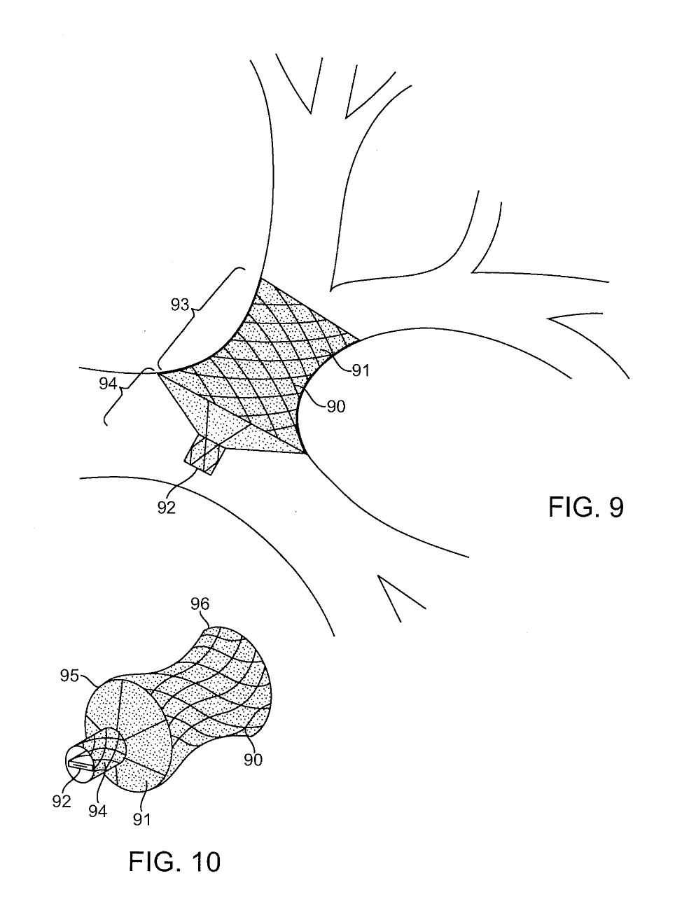

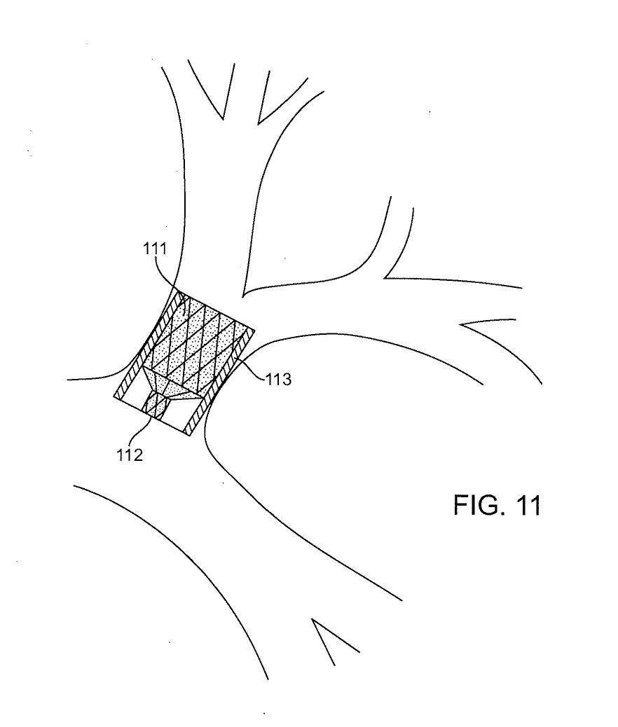

FIGS. 13 and 14 show yet another embodiment of a bronchial isolation device. The device comprises a self-expanding retainer 130, a deformable membrane 131, a one-way valve 132, and valve protector section 134 and a distal retainer section 133. These components are assembled in a substantially different configuration than previous embodiments. The tubular distal retainer 133 is sized and shaped to be implanted into the next most proximal bronchial passageway from the bronchial passageway 135 that is targeted for isolation. The valve protector section 134 incorporates the one-way valve 132, and this assembly is connected to and branches off of the side of the distal retainer 133 and extends into the bronchial passageway 135 targeted for isolation. Thus, the valve protector section 134 is positioned between opposite ends of the distal retainer 133 such that the valve protector section 134 branches out of the distal retainer 133.

In order for the device to work effectively, airflow between the device and the bronchial passageway wall is prevented during inhalation. This can be done by allowing the self-expanding valve protector section 134 to expand and seal against the bronchial passageway 135 that is targeted for isolation, by allowing both the proximal edge 136 and the distal edge 137 of the distal retainer 133 to expand and seal against the walls of the next most proximal bronchial passageway from the bronchial passageway 135 that is targeted for isolation, or both.

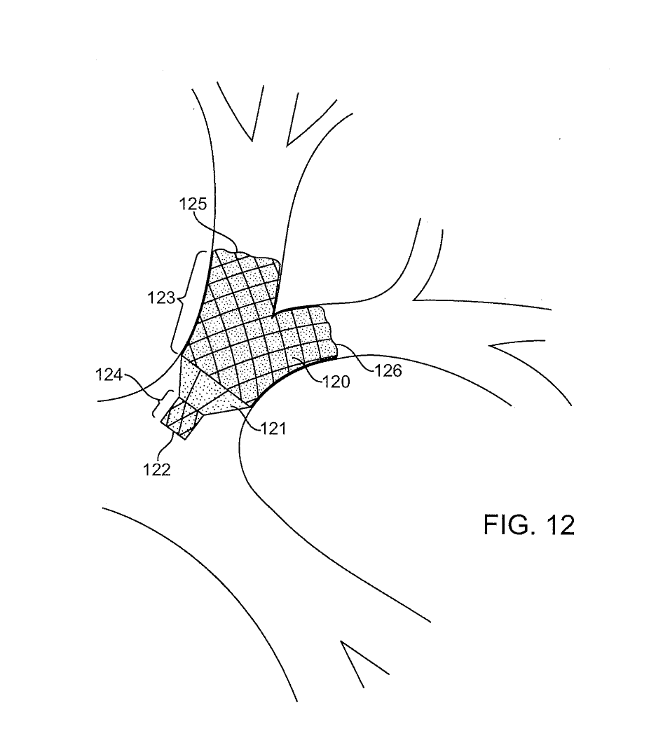

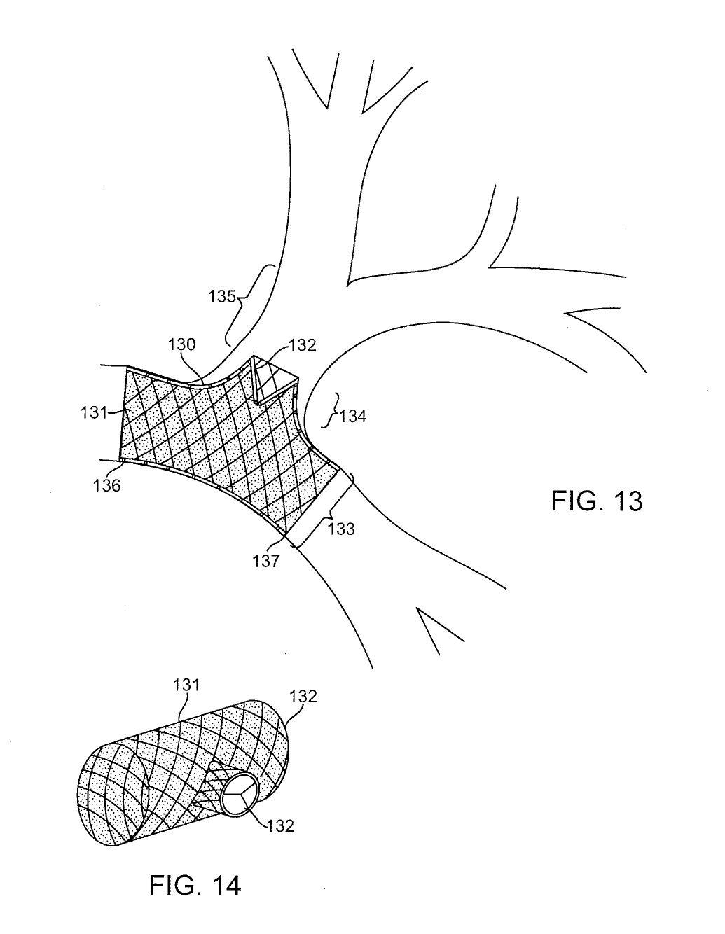

FIGS. 15 and 16 show yet another embodiment of a bronchial isolation device. The device includes a first frame formed of a self-expanding retainer 150 that is positioned remotely from a valve 152. The device is anchored in place by implanting the self-expanding retainer 150 into one of the bronchial passageways that is distal to the bronchial passageway 155 that is targeted for isolation. This retainer 150 may or may not be covered with a deformable membrane. A one-way valve 152 is bonded to or otherwise assembled to a second frame formed of a flexible disk 151 that is attached to the retainer 150 with an extender, such as a tether 154 that may optionally have an elastic section 153. The optional elastic section 153 biases that flexible disk 151 against the ostium of the target bronchial passageway 155 causing the flexible disk 151 to seal against the ostium and prevent passage of inhaled gas or fluid. The one-way valve 152 is coupled to the flexible disk 151 in various manners. The one way valve 152 allows exhaled gas or fluid to pass out of the region targeted for isolation. The elastic section 153 can be comprised of a metal or plastic coil spring, an elastic material such as silicone, or any other material or composition that would bias the flexible disk 153 against the ostium of the target bronchial passageway 155.

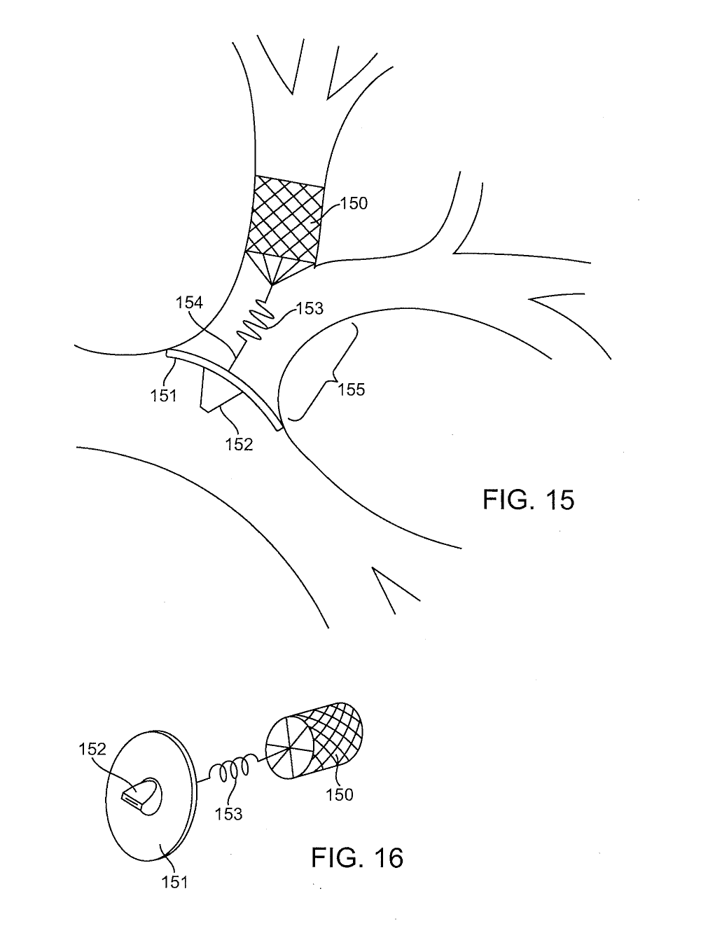

FIG. 17A shows yet another embodiment of a bronchial isolation device. As with the embodiment shown in FIGS. 15 and 16, the device is anchored in place by implanting a first frame formed of a self-expanding retainer 170 into one of the bronchial passageways that is distal to the bronchial passageway 174 that is targeted for isolation. This retainer 170 may or may not be covered with a deformable membrane. A bronchial isolation device 171 having a second frame, similar to the embodiment shown in FIG. 1B, is attached to the retainer 170 with a tether 173. The tether 173 may be elastic or inelastic. The bronchial isolation device 171 is implanted in the target bronchial passageway 174. The tether, which is connected to the retainer 170, stabilizes the bronchial isolation device and prevents it from rotating, migrating or otherwise moving from the target bronchial passageway 174. As before, the bronchial isolation device 171 prevents the passage of gas or fluid in the inhalation direction into the lung portion targeted for isolation, and allows the passage of gas or fluid through the one-way valve 172 in the exhalation direction.

FIG. 17B shows yet another embodiment of a bronchial isolation device. This embodiment is similar to the embodiment of FIG. 17B. However, the bronchial isolation device differs in configuration. As in the previous embodiment, the device is anchored in place by implanting a first frame formed of a self-expanding retainer 170 into one of the bronchial passageways that is distal to the bronchial passageway 174 that is targeted for isolation. This retainer 170 may or may not be covered with a deformable membrane. A bronchial isolation device 175 is attached to the retainer 170 with a tether 173. The tether 173 may be elastic or inelastic. The bronchial isolation device 175 is implanted in the target bronchial passageway 174. The bronchial isolation device 175 includes a second frame or body that defines an internal passageway or lumen through which fluid can flow. A one way valve 172 regulates fluid flow through the passageway. One or more flange-like seal members 177 extend radially outward from the device such that outer edges or regions of the seal members 177 seal against the wall of the bronchial passageway 174 to prevent fluid flow around the device.

The tether 173, which is connected to the retainer 170, stabilizes the bronchial isolation device 175 and prevents it from rotating, migrating or otherwise moving from the target bronchial passageway 174. As before, the bronchial isolation device 175 prevents the passage of gas or fluid in the inhalation direction into the lung portion targeted for isolation, and allows the passage of gas or fluid through the one-way valve in the exhalation direction.

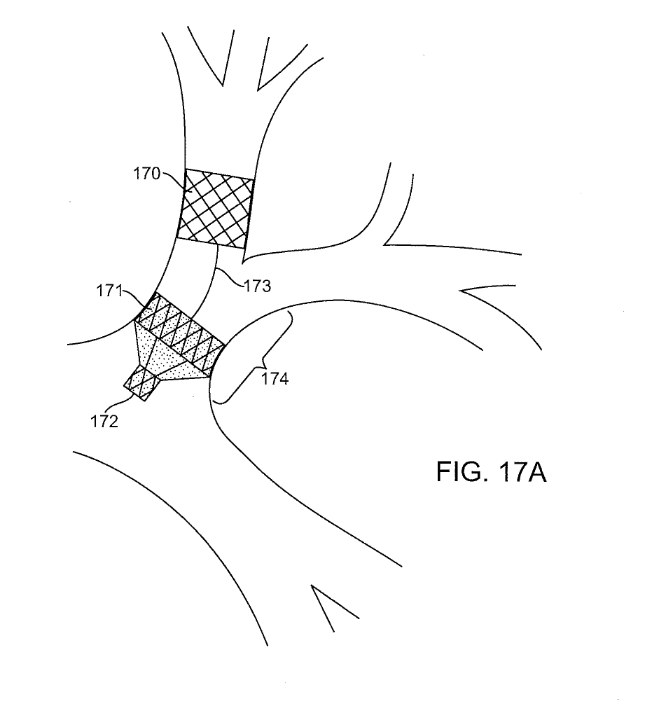

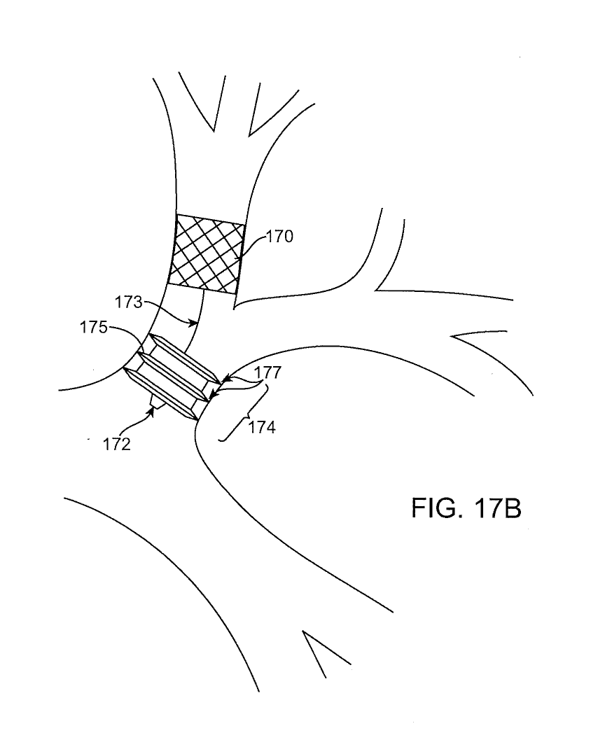

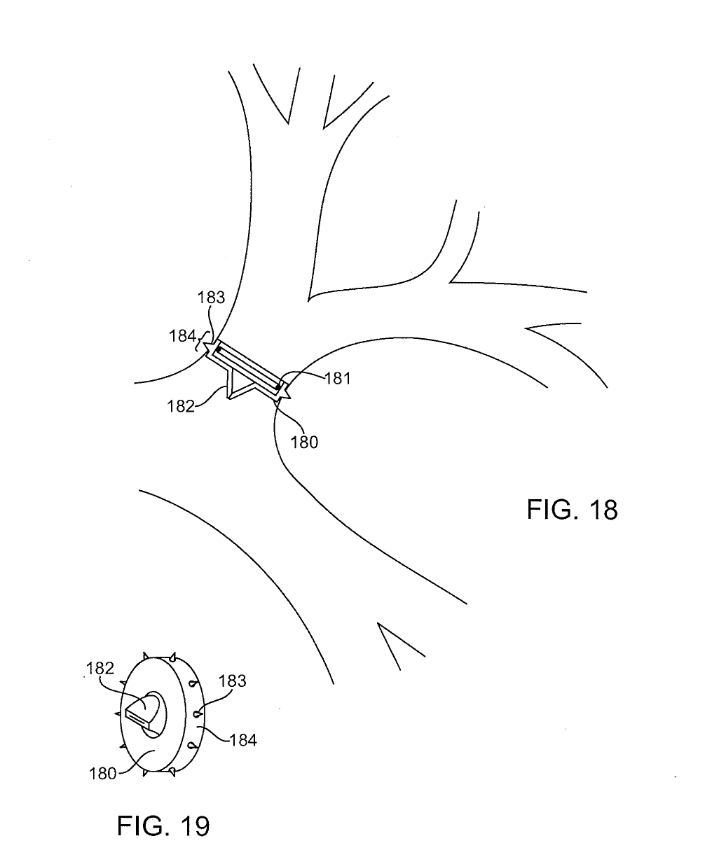

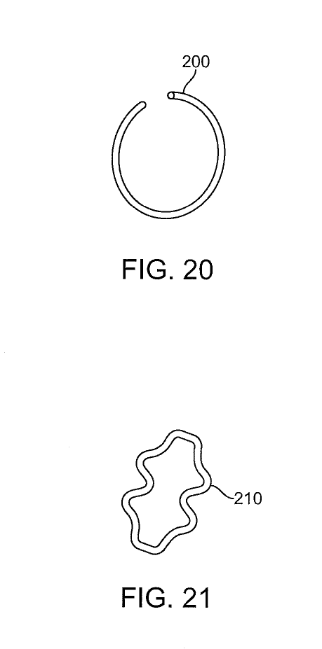

FIGS. 18 and 19 show yet another embodiment of a bronchial isolation device. The device is comprised of a deformable disk frame 180 that has a one-way valve 182 mounted in the center. The outer rim 184 of the disk frame 180 has one or more retention members, such as prongs 183, that sink into or otherwise anchor in the bronchial passageway tissue to hold the device in place and to prevent migration, movement or rotation of the device following placement. In order to bias the outer rim 184 of the disk frame 180, and the retention prongs 183, into close contact with the bronchial passageway wall, a resilient expansion member, such as a resilient ring 181, is positioned inside the outer rim 184. The expansion ring 181 can be made of Nitinol or any other resilient metal or other material. The expansion ring 181 can be a simple ring, a split ring 200 as shown in FIG. 20, a serpentine or wavy ring 210 as shown in FIG. 21, or any other construction that will allow the expansion ring 181 to be resiliently deformable. It can be incorporated into the outer rim 184, bonded to the outer rim 184, snapped inside the outer rim 184 or combined in any other fashion. The device is compressed for placement into the bronchial passageway, and then released to expand into contact with the bronchial passageway wall and to press the retention prongs 183 into the bronchial passageway tissue.

As mentioned previously, all of the bronchial isolation device embodiments shown in the figures and described above are one-way valve bronchial isolation devices. It should be obvious that all of these embodiments could also be two-way valve isolation devices or occluding isolation devices.

What is claimed:

1. A flow control device for a bronchial passageway, comprising:
a valve member that regulates fluid flow through the flow control device;
a frame coupled to the valve member, the frame including:
(a) a distal retainer region being formed of a plurality of interconnected struts configured to engage an interior wall of the bronchial passageway to retain the flow control device in a fixed location therein, the retainer region being movable from a contracted state suitable for introduction into the bronchial passageway to an expanded state suitable for engaging the interior wall of the bronchial passageway; and
(b) at least one stabilization structure having a tip that extends distally past a distal edge of the distal retainer region, the stabilization structure sized and shaped to achieve stabilization of the position of the device in the bronchial passageway, wherein the stabilization structures rest against and do penetrate the bronchial wall; and
a membrane covering at least a portion of the retainer region, wherein at least a portion of the flow control device forms a seal with the interior wall of the bronchial passageway when the flow control device is implanted in the bronchial passageway.

2. The device of claim 1, wherein the stabilization structure comprises a barb with a sharpened tip.

3. The device of claim 1, wherein the stabilization structure comprises an arm with a rounded tip.

4. The device of claim 1, wherein the frame includes a plurality of stabilization structures that are interspersed around a circumference of a distal edge of the frame.

5. The device of claim 1, wherein the frame further includes a valve protector region that at least partially surrounds the valve member.

6. The device of claim 5, wherein the membrane covers the valve protector region.

7. A flow control device for a bronchial passageway, comprising:
a valve member that regulates fluid flow through the flow control device;
a frame coupled to the valve member, the frame including:
(a) a distal retainer region formed of a plurality of interconnected struts configured to engage an interior wall of the bronchial passageway to retain the flow control device in a fixed location therein, the retainer region being movable from a contracted state suitable for introduction into the bronchial passageway to an expanded state suitable for engaging the interior wall of the bronchial passageway, wherein the distal retainer region includes an elongated section that is sufficiently long to extend into a distal bronchial passageway that branches from a bronchial passageway in which the device is implanted; and
a membrane covering at least a portion of the distal retainer region not including the elongate section, wherein at least a portion of the flow control device forms a seal with the interior wall of the bronchial passageway when the flow control device is implanted in the bronchial passageway.

8. A device as in claim 7, wherein the membrane seals with the wall of the bronchial passageway in which the valve protector region is implanted.

9. A device as in claim 7, wherein the valve protector region is formed of a plurality of interconnected struts.

10. The device of claim 7, wherein the frame further includes a valve protector region that at least partially surrounds the valve member.

11. A method of implanting a fluid flow control device in a bronchial passageway, comprising:
providing a flow control device having a valve, a retainer, and a seal; and
implanting the flow control device in a bronchial passageway such that a proximal portion of the retainer anchors against a wall of a first bronchial passageway and a distal portion of the retainer is positioned in a second bronchial passageway that branches from the first bronchial passageway, wherein the seal covers only the proximal portion of the bronchial passageway.

12. A method as in claim 11, further comprising permitting fluid to flow into the flow control device from a third bronchial passageway, wherein the fluid flows into the flow control device through the distal portion of the bronchial passageway.

13. A flow control device for a bronchial passageway, comprising:
a frame having a first end and a second end, the frame defining an internal lumen having a first opening at the first end and a second opening at the second end that both communicate with the lumen;
a seal covering at least a portion of the frame; and
a valve positioned on the frame between the first end and the second end, wherein the valve regulates fluid flow into the lumen at a location between the first end and the second end.

14. A flow control device as in claim 13, wherein the frame is formed of interconnected struts.

15. A flow control device as in claim 13, wherein the seal is a membrane.

16. A flow control device for a bronchial passageway, comprising:
a first frame adapted to anchor against a wall of a first bronchial passageway, the first frame defining a lumen through which fluid can flow;
a seal member coupled to the first frame and adapted to seal against the first bronchial passageway;
valve member that regulates fluid flow through the lumen, the valve member coupled to the first frame;
a second frame adapted to anchor against a wall of a second bronchial passageway; and
a tether connecting the first frame to the second frame.

17. A flow control device as in claim 16, wherein at least one of the first and second frames is formed of a plurality of interconnected struts.

18. A flow control device as in claim 16, wherein the seal member comprises at least one flange that extends radially outward from the first frame.

19. A flow control device as in claim 16, wherein the first frame comprises a flexible disk that seals against an ostium of the first bronchial passageway.

20. A flow control device as in claim 16, wherein the second frame is covered by a seal member.

21. A flow control device as in claim 16, wherein the tether is at least partially elastic
